(19) United States
(12) Patent Application Publication
(10) Pub. No.: US 2013/0059358 A1
(43) Pub. Date: Mar. 7, 2013

(54) IN-SITU ELECTRICAL STIMULATION OF BIOCONVERSION OF CARBON-BEARING FORMATIONS

(71) Applicants: Robert A. Downey, Centennial, CO (US); Song Jin, Fort Collins, CO (US); William J. Brown, Denver, CO (US); Paul H. Fallgren, Highlands Ranch, CO (US)

(72) Inventors: Robert A. Downey, Centennial, CO (US); Song Jin, Fort Collins, CO (US); William J. Brown, Denver, CO (US); Paul H. Fallgren, Highlands Ranch, CO (US)

(73) Assignee: CIRIS ENERGY, INC., Centennial, CO (US)

(21) Appl. No.: 13/696,646

(22) PCT Filed: May 11, 2011
(86) PCT No.: PCT/US11/00819

§ 371 Date: Nov. 7, 2012

Related U.S. Application Data
(60) Provisional application No. 61/333,330, filed on May 11, 2010.

(51) Int. Cl. C12P 5/02

(52) U.S. Cl. 435/167

(57) ABSTRACT

Methods of stimulating microbial consortia, such as microbial consortia in a geological formation, such as comprising methanogens and other bacteria, for producing methane and other fuels or fuel precursors from coal or other carbonaceous materials, are disclosed along with methods for increasing bioconversion of carbonaceous materials, such as coal, into methane and other useful hydrocarbon products, wherein the consortia respond to electrical stimulation, either physical or chemical.

This application claims priority of U.S. Provisional Application 61/333,330, filed 11 May 2010, the disclosure of which is incorporated herein by reference in its entirety.

### FIELD OF THE INVENTION

The present invention relates to the production of methane, carbon dioxide, gaseous and liquid hydrocarbons, and other valuable products from subterranean carbon bearing formations, in-situ, by electrical stimulation of microbial consortia in such formations.

### BACKGROUND OF THE INVENTION

Methanogenesis (also known as biomethanation) is the production of methane by microbes. The production of methane is an important and widespread form of microbial metabolism. Methanogenesis in microbes is a form of anaerobic respiration and represents the end reaction in the decay of organic matter. These reactions result in the depletion of electron acceptors (such as oxygen) and the accumulation of small organics, such as hydrocarbons, especially methane, as well as gases like hydrogen and carbon dioxide. During such processes, fermentation breaks the larger organics while methanogenesis removes the smaller materials, such as hydrogen, carbon dioxide and small organic molecules.

Electrical bio-stimulation is the supply of electrons to stimulate the growth of microbes. All organisms require electron donors and acceptors, and the electrons may be provided either in chemical form or by direct electrochemical means. Electricity has been used to stimulate microbial metabolism for many years. However, electrical stimulation of microbes in a subterranean carbon-bearing formation for the purpose of the bioconversion of coal or other carbonaceous materials to methane and other gaseous or liquid hydrocarbons useful as fuels or fuel precursors has not been reported.

### BRIEF SUMMARY OF THE INVENTION

In one aspect, the present invention relates to a bioconversion process, comprising introducing electrical energy into a carbon bearing deposit, such as a coalseam or other carbonaceous deposit, and subsequently removing formed product from the deposit. In different embodiments, the source of the electrical energy is physical or chemical or both (such as a physical and/or chemical source of electrons) and the formed product is a fuel, such as methane, or a fuel precursor (i.e., a material readily converted into a fuel).

In one application, this method includes injection of a fluid into a deposit, together with or separately from said electrical energy, as well as subsequent removal of the fluid, either together with the formed products or before or after removal of the formed products.

In one embodiment, the introduced fluid contains nutrients that promote and/or support the growth of microbes present in the carbon bearing deposit. In additional embodiments, the fluid contains chemicals that solubilize at least a portion of the carbonaceous material in the deposit, especially as a means of facilitating bioconversion.

In an alternative embodiment, the injected fluid contains microbes, such as non-indigenous microbes, to bioconvert carbonaceous material of the formation to methane or other useful fuels or fuel precursors.

In separate examples, the delivery of electrical energy is continuous or is intermittent. In other examples, the injection of nutrients and chemicals is continuous or intermittent. In some cases, the electrical energy and chemicals are introduced intermittently but in a staggered manner, so that electrical energy and nutrients are administered alternately.

In a preferred embodiment of the inventive method, wellbores are extended from the surface into a carbon bearing deposit. In one embodiment, such wellbores comprise injection wells, production wells, and electrical energy delivery wells, preferably extended from the surface into the carbon bearing deposit horizontally, directly above or below one another. In at least one such embodiment, one or more wellbores are utilized solely for the delivery of electrical energy and one or more wellbores are utilized for the injection and production of fluids, gases, nutrients and chemicals.

In one example of the methods or processes of the invention, materials that increase the efficiency of electrical energy delivery into the carbon bearing deposit are added to fluids, nutrients, gases and chemicals injected into the carbon bearing deposit.

In another example, the injected fluid flows from an injection well to a plurality of production wells and/or the distribution of fluid flow from the injection well to the production wells is controlled by controlling the pressure difference between the injection well and the production wells. The distribution may also be controlled through the formation to increase the total production of methane and other fuels or fuel precursors.

In at least one embodiment, carbon dioxide is added to water, nutrients, chemicals and gases that are injected into said carbon bearing deposit and is converted into methane by indigenous and/or non-indigenous methanogenic consortia in the formation and then recovered from the carbon-bearing deposit, such as from coal in a coalseam.

### BRIEF DESCRIPTION OF THE DRAWING

FIG. 1 shows one means by which electrical energy is directed into a coalseam in order to stimulate the bioconversion of carbonaceous material to methane. Here, 1 represents an injection well while 2, 3, 4 and 5 are production wells.

### DEFINITIONS

As used herein, the term “bioconversion” refers to the conversion of carbonaceous molecules (such as in a carbon-bearing formation, for example, coal in a coalseam, into methane and other useful gases and liquid products, preferably by indigenous microbes in the deposit or by non-indigenous microbes introduced into the deposit. Such bioconversion is stimulated to occur by the application of electricity from a chemical or physical source.

As used herein, “coal” refers to any of the series of carbonaceous fuels ranging from lignite to anthracite. The members of the series differ from each other in the relative amounts of moisture, volatile matter, and fixed carbon they contain. Coal is comprised mostly of carbon, hydrogen and entrained water, predominantly in the form of large molecules having numerous double carbon bonds. Low rank coal deposits are mostly comprised of coal and water. Energy can be derived from the combustion of carbonaceous molecules, such as coal, or carbonaceous molecules derived from the solubilization of coal molecules. Of the coals, those containing the largest amounts of fixed carbon and the smallest amounts of moisture and volatile matter are the most useful.

As used herein, the term “solubilizing” or “solubilized” refers to a process whereby the very large hydrocarbon molecules that comprise coal or other carbonaceous material are reduced to much smaller hydrocarbon molecules or compounds by the application of one or more chemicals that can cleave carbon bonds and other chemical bonds of coal molecules and react with the chemicals to form smaller hydrocarbon molecules that are then be biologically converted to methane, carbon dioxide and other useful gases. Solubilization for the purposes of the invention means the conversion of a solid carbonaceous material, such as coal, to a form of carbon that is in solution with water, and more specifically a form of carbon comprised of compounds that are soluble in water and capable of passing through a 0.45 micron filter.

As used herein, the term “salts or esters of acetic acid” means the conjugate base of acetic acid, where the acetate ion is formed by deprotonation of acetic acid, or an organic compound with the general formula CH₃CO₂R, where R is an organic group.

As used herein, the term “acetate” refers to the salt wherein one or more of the hydrogen atoms of acetic acid are replaced by one or more cations of a base, resulting in a compound containing the negative organic ion of CH₃COO—. Said term also refers to an ester of acetic acid. In accordance with the invention, said salts or esters of acetic acid are optionally mixed with water. In one preferred embodiment, the salts or esters of acetic acid are used in admixture with water. It is to be appreciated that when such acetate salts are employed using a water solvent, some acetic acid may be formed (depending on the final pH) and will participate in the solubilization process. For purposes of the invention, a similar definition is to be understood where a salt of another carboxylic acid, such as benzoic acid, is used for like purposes.

As used herein, the term “aromatic alcohol” means an organic compound having the formula ROH, wherein R is a substituted or unsubstituted aromatic group, which aromatic group may be a monocyclic ring or a fused ring. In one embodiment, the aromatic group R is unsubstituted. In another embodiment, R is substituted with one or more of a hydrocarbon group and/or an —OH group(s). In some embodiments, the —OH is present on the aromatic ring, or is present in a substituent of said ring or both.

The terms “biogasification” and “methanogenesis” are used herein essentially interchangeably.

As used herein, the phrase “microbial consortium” refers to a microbial culture, including a natural assemblage, containing 2 or more species or strains of microbes, especially one in which each species or strain benefits from interaction with the other(s).

As used herein, the term “useful product(s)” refers to a chemical obtained from a carbonaceous material, such as coal, by bioconversion and includes, but is not limited to, organic materials such as hydrocarbons, for example, methane and other small organics, as well as fatty acids, that are useful as fuels or in the production of fuels, as well as inorganic materials, such as gases, including hydrogen and carbon dioxide.

### DETAILED DESCRIPTION OF THE INVENTION

The present invention relates to a method for stimulating microbes and/or microbial consortia within a carbon-bearing geological formation to increase the bioconversion of carbonaceous materials present in such formation, including materials that are exogenous materials introduced into the formation. Such formations are typically subterranean formations, for example, those known as “coal seams” or “coal beds,” that contain carbonaceous materials that can be bioconverted into methane and other gases and liquid products useful as fuels, or fuel precursors (for example, fatty acids, hydrocarbons and any molecules readily converted into methane, and the like) that are then converted to useful fuels by further reactions well known in the industry.

In particular, such method comprises introducing electrical energy (i.e., a physical or chemical source of electrons) into a carbon bearing deposit, such as a coalseam, with or without concurrent fluid injection, and subsequently removing formed product (and any injected fluid or fluids) from the deposit. The microbial consortia, especially those that include methanogens, are the agents that convert carbon-bearing molecules, such as acetate and carbon dioxide, to methane and other fuels and fuel precursors.

In accordance with the foregoing, useful geological formations include mines, river beds, ground level fields and the like, especially where these are rich in carbon-containing materials, for example, a coalseam.

Preferably, the coal-bearing deposit is a coal seam, including where the coal is bituminous coal, lignite or any form or rank of coal, ranging from brown coal to anthracite, based on increasing carbon content. The lowest in carbon content, lignite or brown coal, is followed in ascending order by sub-bituminous coal or black lignite (a slightly higher grade than lignite), bituminous coal, semi-bituminous (a high-grade bituminous coal), semi-anthracite (a low-grade anthracite), and anthracite. All are useful in the methods of the invention.

In preferred embodiments, such conversion uses microbial consortia, which may be indigenous or may be intentionally introduced into the formation, that respond positively to electrical bio-stimulation to produce methane and other useful products. Such methods optionally include a prior or contemporaneous step of solubilization of the carbonaceous contents of the formation as a means of facilitating concurrent and/or subsequent bioconversion.

In accordance with the invention, the bioconversion of coal carbon to methane is increased by the delivery of electrons, in addition to injected nutrients and other chemicals, some of which improve the susceptibility of coal for microbial conversion to methane and/or carbon dioxide. The delivery of electrons is controlled by adjustments to the flow of electrons as current or voltage, by adjustments to the location and operation of the wellbores, and by adjustments to the flow of nutrients and other chemicals, including electron donors such as hydrogen, metals, hydrocarbons and the like.

Carbon dioxide is converted to methane by the action of methanogens and in accordance with the invention the carbon dioxide to methane conversion rate by such methanogens is increased when stimulated with electrons. The chemical equation for the conversion of carbon dioxide to methane by microbes is:

CO₂+8H⁺+8e⁻→CH₄+2H₂O

In one embodiment, the present invention provides a method for converting carbon dioxide into methane using indigenous microbes of a carbon-rich formation, and/or non-indigenous microbes introduced into a formation, that have been electrically stimulated, such as by a chemical source of electrons. Alternatively, carbon dioxide is introduced into subterranean formations with, or followed by, electrical bio-stimulation according to the invention. On a large scale, these methods efficiently convert carbon dioxide (a defined greenhouse gas) into a useful energy product, such as methane or other biofuels.

In one embodiment, the stimulation of methanogenic consortia and production of methane and other valuable gases and hydrocarbons is further enhanced by the injection of certain non-indigenous microbial species, along with nutrients and chemicals, with the introduction of a source of electrons.

In certain embodiments, the bioconversion is effected by one or more bioconversion agents. The bioconversion agents include facultative anaerobes, such as those of the genus Staphylococcus, Escherichia, Corunebacterium and Listeria, acetogens, for example, those of the genus Sporomusa and Clostridium, and methanogens, for example, those of the genus Methanobacterium, Methanobrevibacter, Methanocalculus, Methanococcoides, Methanococcus, Methanocorpusculum, Methanoculleus, Methanofollis, Methanogenium, Methanomicrobium, Methanopyrus, Methanoregula, Methanosaeta, Methanosarcina, Methanophaera, Methanospirillium, Methanothermobacter, and Methanothrix. Bioconversion agents also include eukaryotes, such as fungi.

For example, U.S. Pat. No. 6,543,535 and U.S. Published Application 2006/0254765 disclose representative microorganisms and nutrients, and the teachings thereof are incorporated by reference. Suitable stimulants may also be included,

Additives to the injection fluid include major nutrients, vitamins, trace elements (for example, B, Co, Cu, Fe, Mg, Mn, Mo, Ni, Se, W, Zn as a non-limiting group) and buffers such as phosphate and acetate buffers. Suitable growth media may also be included. In practicing the invention, it may be necessary to first determine the nature of the microbial consortium present in the coal deposit in order to determine the optimum growth conditions to be used as part of the inventive process.

Where bioconversion is accomplished by indigenous (or endogenous) microbial consortia, the above-recited nutrients are advantageously introduced prior to electrical stimulation of bioconversion. Where bioconversion is accomplished by non-indigenous microbes (such as exogenously introduced microbes or microbial consortia), nutrients may be introduced before, during or after introducing the exogenous microbes.

The bioconversion is operated under conditions effective to bioconvert the treated carbonaceous material and/or products obtained from it using microbes. Useful bioconversion agents include facultative anaerobes, acetogens, methanogens and fungi. In exemplary embodiments, bioconversion includes formation of hydrocarbons such as methane, ethane, propane; and carboxylic acids, fatty acids, acetate, carbon dioxide.

In suitable embodiments, the bio-electrical stimulation of endogenous and/or exogenous microbes or microbial consortia within a geological formation comprises introducing electrons, with or without the injection of water and/or other chemicals into a geological formation, such as a coalseam and collecting unused electrons so as to complete an electrical circuit, with subsequent recovery of formed products and/or injected materials from the formation.

The flow of the introduced electrons is controlled to such an extent that the growth of the microbes in the coal seam and the conversion of carbonaceous material, such as coal, and, optionally, another carbon source introduced, such as carbon dioxide, is increased and maintained at a selected level.

Electrical conductivity is generally proportional to water salinity. Carbonaceous deposits that have large amounts of dissolved ions, and particularly metal salt ions such as sodium and chloride, conduct electrical current, or allow the flow of electrons, more easily than those having low amounts of dissolved ions. The resistance to current flow between an anode and cathode placed into, or in electrical contact with, a formation having a high salinity level will therefore be lower than for one with a low salinity level. For example, in low salinity coalseams, conductivity, or the ability of electrical energy to flow through the water, is adjusted by the addition of electrically conductive materials that optionally also support or enhance the growth of indigenous and/or non-indigenous, i.e., endogenous and/or exogenously introduced, methanogenic consortia. In one embodiment of the invention, iron nanoparticles and/or soluble salts are added to water injected into a coalseam to increase the conductivity of the water and provide iron that enhances electron transfer by the consortia.

The predominant form of void space in geological formations, such as coalseams, is in fractures, which may range in aperture or width from sub-microns to millimeters, and in length from microns to hundreds of feet. Many (even most) of these fractures are interconnected and thus constitute a hydraulic circuitry through which both fluids and gases, and electrical energy, flow. Coalseams have a high degree of compressibility in relation to other formations, such as sandstones and shales, and therefore the solid volume, and void space, within a coalseam is adjusted by increasing or decreasing the fluid pressure within the coalseam. Operation of the methods of the invention in a coalseam at a fluid pressure above initial or hydrostatic conditions and at optimum net effective stress may increase inter-well permeability as the process proceeds, may increase the amount or volume of fluid in coalseam, may increase the number of microbes that may exist in the coalseam water, and may increase the volume or number of electrons that are provided to the microbes and thus may increase the efficiency of the process.

In accordance with the invention, subterranean carbon-bearing formations may at any time be saturated with fluids, such as liquids and/or gases, and such saturations also affect the net effective stress on the formations. The permeability of gases and liquids in the subterranean formation is also dependent upon their saturations, and thus by purposefully increasing the pressure within the subterranean formation well above its initial condition, to an optimum point, and maintaining that pressure continuously, the flow of fluids, nutrients, microbial consortia and generated methane, carbon dioxide and hydrocarbons are optimized.

The maximum pressure in which the process is operated is limited by the point at which the fluid pressure in the subterranean formation exceeds the tensile strength of the formation, causing fractures to form and propagate in the formation, in either a vertical or horizontal plane, as determined by Poisson's ratio. These pressure-induced fractures often form large fluid channels through which the injected fluids nutrients and microbial consortia and generated methane flow, thus reducing or inhibiting distribution of fluid pressure and reduction of net effective stress throughout the subterranean formation.

Operation of the conversion process at a subterranean formation at a pressure point above initial or hydrostatic conditions and at optimum net effective stress enables better determination of inter-well permeability trends and changes in inter-well permeability as the process proceeds. The bioconversion of solid coal or shale to methane gas reduces the solid volume of the coal or shale along the surfaces and increases the fracture aperture and pore diameter of the relevant porosities that, in turn, increase the permeability of the subterranean formation and the efficiency of the conversion process.

Many carbon-bearing subterranean formations have multiple types of porosity, or pore space, a function of the type of material they are comprised of and the forces that have been and are exerted upon them. Many coal seams, for example, have dual or triple porosity systems, whereby pore spaces exist as fractures, large matrix spaces and/or small matrix spaces. These pore spaces vary substantially across an area, often exhibit directional trends or orientations, and are often variable in their vertical orientation within the subterranean formation. The permeability of subterranean formations can also vary substantially horizontally and vertically within a given subterranean environment. Given sufficient geological and geophysical data, a number of characteristics of a subterranean formation such as thickness, areal extent, depth, slope, saturation, permeability, porosity, temperature, formation geochemistry, formation composition, and pressure are ascertained in order to form a 3-dimensional mathematical model of the subterranean formation.

In accordance with the invention, in situ bioconversion of carbon-bearing subterranean formations to carbon dioxide, methane and other hydrocarbons is performed using indigenous and/or intentionally introduced non-indigenous methanogenic consortia via the introduction of microbial nutrients, methanogenic consortia, chemicals and electrical energy, utilizing a comprehensive mathematical model that fully describes the geological, geophysical, hydrodynamic, microbiological, chemical, biochemical, geochemical, thermodynamic and operational characteristics of such systems and processes.

The amount of such bioconversion component products that are produced, and the rate of such production, is a function of several factors, including but not necessarily limited to, the specific microbial consortia present, the nature or type of the carbon-bearing formation, the temperature and pressure of the formation, the presence and geochemistry of the water within the formation, the availability and quantity of nutrients required by the microbial consortia to survive and grow, the presence or saturation of methane and other bioconversion products or components, as well as other factors.

The rate of carbon bioconversion is proportional to the amount of surface area available to the microbes utilized in the conversion process, the population of the microbes and the movement of nutrients into the deposit and bioconversion products extracted from the deposit as the deposit is depleted. The amount of surface area available to the microbes is proportional to the percentage of void space, or porosity, of the subterranean formation and the permeability (a measure of the ability of gases and fluids to flow through the subterranean formation) is in turn proportional to its porosity. All subterranean formations are to some extent compressible, i.e., their volume, porosity, and permeability is a function of the net stress upon them. Their compressibility is in turn a function of the materials, i.e., minerals, hydrocarbon chemicals and fluids, the porosity of the rock and the structure of the materials, i.e., crystalline or non-crystalline.

The methods of the invention take advantage of the preceding factors so that stimulation of methanogenic consortia and the production of methane within a formation, such as a coalseam, are independently adjusted or controlled or directed by the injection and/or production of fluids, nutrients, chemicals, electrically conductive materials and electrical energy into and out of the formation by means of injection and production wells, under varying conditions of pressure and flow rate, which in turn causes changes to the formation porosity, permeability, movement of fluids and gases through the formation, and the electrical conductivity properties of the formation. For example, adjusting the injection rate and pressure into a coalseam and/or controlling release of fluid from at least one production well increases or decreases the volume of fluids in the coalseam, the permeability of the coalseam and the effective permeability of the coalseam, and thus the electrical conductivity and current flow within the coalseam.

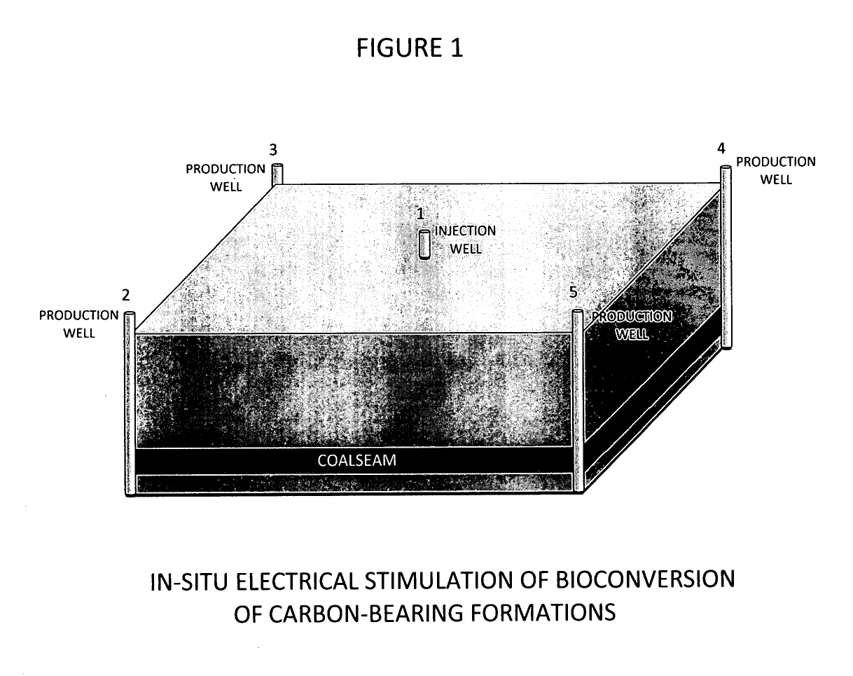

In one non-limiting example, where the formation is a coalseam, the stimulation of microbes, including methanogenic consortia, and production of methane and other valuable gases and hydrocarbons is further enhanced or optimized by the utilization of an array of wellbores or hydraulic and/or electrical conduits into a geological formation, such as a coalseam or other carbon-rich deposit. In one such example, a group of wellbores (see FIG. 1), oriented vertically and/or horizontally into the coalseam are utilized as anodes and another group of wellbores located near the first group of wellbores are utilized as cathodes. Alternatively, two or more wellbores are provided so as to direct current flow through the coalseam, while other wellbores are provided to inject water, nutrients, chemicals and other materials into the coalseam and still other wellbores are provided to produce gases, water and other materials from the coalseam.

In one embodiment, the stimulation of methanogenic consortia and production of methane and other valuable gases, including other hydrocarbons, is further enhanced by the utilization of wellbores that are oriented horizontally or at angles other than vertical, thus increasing the surface area of the coal seam reservoir exposed to the injection and production of fluids, chemicals, materials and gases, and the application of electrical energy into the coalseam.

In one embodiment, the stimulation of methanogenic consortia and production of methane and other valuable gases and hydrocarbons is further enhanced by the application of electrical energy in either a continuous or non-continuous manner, and by varying the voltage and/or amperage of the applied electrical energy, so as to optimize the process.

In one embodiment, electrons are introduced by means of an electrically conductive circuit and discharge point (called an anode) via a wellbore and into the coalseam, either directly into the coalseam or into water injected into the coalseam. The electrons flow through the coalseam to one or more electrically conductive points constructed within one or more wellbores, known as a cathode or cathodes. Electrons are thereby provided to microbes contained within water in the void spaces in the coalseam, that may be contained in the water and/or may be attached to the surface of the solid coal material but in contact with the water. In one example, the flow of electrons through the coalseam and thus to the microbes is controlled by means of the construction of the electrical circuit and its operation. For example, the voltage and current flow may be adjusted by means well known in the art for controlling the supply of electrical energy. The flow of electrons is further adjusted by the location and configuration of the anode- and cathode-containing wellbores.

In a non-limiting application of the invention, at least two wellbores, or other means of communication, are established between a buried carbonaceous formation, such as a coalseam, and the surface or ground level, and the wellbores are constructed to enable the circulation of fluids between the wellbores, electrical isolation within the formation, and the formation of a closed electrical circuit between the wellbores. Such electrical energy may derive from a physical source, such as an electrode, for example, one that is attached to a battery or other electrical generator, or may derive from an electrochemical source, such as chemical-containing electrodes, or such electrical energy may be electrochemical in nature, such as redox reagents that transfer electrons in a redox reaction.

In one embodiment, electrical conduits are inserted into the wellbores by means that enable the delivery of electrons into and from the carbonaceous formation and isolated from other formations. Fluid containing chemicals useful for the bioconversion of coal to methane and other products, is injected into the formation and electrical energy is delivered into the formation to provide bioelectrical stimulation of indigenous consortia. The fluid is injected into the formation through one or more injection wells and flows through the formation to reach one or more production wells whereby the injected fluid is withdrawn from the formation along with materials produced by the bioconversion process. Electrical energy is also delivered into the formation through one or more wells, providing electrons for bioelectrical stimulation of microbes in the formation, and flows through the formation to one or more production wells, where at least a portion of the electrical energy is recovered, completing a closed electrical circuit between the wells.

In one embodiment, a coalseam has a very high carbon content, possibly exceeding 70%, and also may have a very high resistance to electrical current flow. The void space, or porosity, in most coalseams is contained in large numbers of fractures, sometimes known as “cleats”, and this void space is usually filled with water. Further, the water filling the void spaces in coalseams usually contains some dissolved mineral content, and as a result has an electrical conductivity much greater than the solid coal in the coalseam. As a result, electrical energy delivered into a coalseam flows almost completely in the water in the porosity and not through the solid coal in the coalseam (or into other formations above or below the coalseam).

As shown in FIG. 1, there is an injection well 1 plus four production wells 2, 3, 4 and 5. The wells are constructed so that an electrically conductive conduit is extended from the surface through the wellbore of each well, enabling the delivery of electrical energy into a carbon-bearing formation, such as a coalseam. Direct current electrical energy flows from an electrical energy delivery source through the conduit extending through the injection wellbore to a cathode and into the carbon-bearing formation, for example, a coalseam, to the anode connected to the electrical conduit extending to the formation in each of the producing wells, and returns to the electrical energy delivery source on the surface. The electrical energy supplies electrons into the formation fluids and onto the surface of the formation solids, supplying electrons that are utilized by indigenous methanogenic consortia to enhance their metabolic function and the generation and production of methane. The amount of electrical energy, in terms of voltage and amperage, may be adjusted from the surface electrical source and/or by adjustment of electrical resistance in the conducting conduits. The carbon-bearing formation, such as a coalseam, and the fluids occupying the pore spaces in the formation, have a resistance to current flow proportional to the conductivity of the fluids and/or solid material in the formation. The flow of electrical energy, in terms of amperage and voltage, is affected by the location and distance between the cathode and anodes.

In preferred embodiments, electrical energy is supplied to the carbon-bearing formation, such as coal, along with chemicals that promote the growth of indigenous methanogenic consortia and the generation and production of methane and other useful products.

In one preferred embodiment, carbonaceous material, such as coal, is bioconverted within a formation by a combination of solubilization of coal by one or more solubilization chemicals, including ester(s), hydroxide(s) and/or peroxide(s), and bioconversion of the solubilization product, using one or more chemicals and/or nutrients and/or vitamins and/or minerals recited herein to promote and/or support the bioconverting microbes.

In other preferred embodiments, electrical energy is supplied to the carbon-bearing formation, such as coal, simultaneously with chemicals that promote the growth of methanogenic consortia and the generation and production of methane and other useful products, as well as chemicals that may solubilize at least a portion of the carbon-bearing formation, thereby promoting the generation and production of methane and other useful products.

The amount of bioconversion products produced by methanogenesis in a carbon-rich formation, such as a coal seam, and the rate of such production, depends on several factors, including but not necessarily limited to, the specific microbial consortia present, the nature or type of the coal seam, the temperature and pressure of the coal seam, the presence and geochemistry of the water within the coal seam, the availability of nutrients required by the microbial consortia to survive and grow, the availability of bio-available carbon, the availability of electrical energy that may stimulate the growth of the microbial consortia, and the presence or saturation of methane and other bioconversion products.

In one or more embodiments of the invention, the permeability of a formation, such as a coal seam, is increased and/or optimized by increasing fluid pressure within at least a portion of the formation, during processes for producing methane and the introduction of electrical energy. In addition, bioconversion of the coal to methane, carbon dioxide, and various hydrocarbons, especially small molecules, is also optimized by increasing one or more of the delivery and dispersal of nutrients into the formation, the delivery and dispersal of microbial consortia in the formation, and the amount of surface area of the formation that is exposed to the microbial consortia. As a result, the removal and recovery of the generated methane, carbon dioxide, and other hydrocarbons from the formation is likewise facilitated.

The rate of carbon bioconversion is proportional to the optimization of electrical stimulation applied to the microbes in a formation, the amount of surface area available to the microbes, to the population of the microbes, and to the movement of nutrients into the system and the movement of bioconversion products from the system. The amount of surface area available to the microbes is proportional to the percentage of void space, or porosity, of a subterranean formation and the ability of gases and liquids to flow through the subterranean formation is in turn dependent on its porosity. All subterranean formations are to some extent compressible. The amount of electrical energy to be applied is at least partially dependent upon the amount of porosity of, or fluid space within, a subterranean formation. Thus, in accordance with the invention, by reducing the net effective stress upon a formation, for example, by increasing the fluid pressure therein, one can improve the formation's permeability, porosity, internal surface area available for bioconversion, and the ability to apply electrical energy through the formation, and thereby move nutrients, microbes and generated methane, carbon dioxide, and small hydrocarbons into and out of the deposit.

In accordance with the invention, the stimulation of methanogenic consortia and production of methane and other valuable gases and hydrocarbons is enhanced by the addition of chemicals that can solubilize carbonaceous materials like coal, providing for more bio-available carbon. In accordance with the methods of the invention, such addition occurs during or prior to the bioconversion process.

Thus, as noted already, the carbonaceous materials are first solubilized in situ by contacting the material with one or more chemicals that break many of the chemical bonds that comprise the contained molecules and thereby serve to solubilize it. These chemicals, used either alone or in combination, are contacted with the carbon-containing material at selected concentrations, temperatures and steps in order to maximize the solubilization process.

Such additives include peroxides, hydroxides, benzoic acids, C1-C4 carboxylic acids, preferably aliphatic acids, most preferably acetic acid, including salts or esters of any of these carboxylic acids, preferably esters such as acetates, that are employed individually, sequentially or in selected combinations and sub-combinations. In preferred embodiments, the latter chemicals are, or include, sodium hydroxide, hydrogen peroxide and/or ethyl acetate. It is to be appreciated that when such acetate salts are employed using a water solvent, some acetic acid will be formed (depending on the final pH) and will participate in the solubilization process.

In one embodiment, the method includes contacting carbonaceous material in a geological formation, preferably one rich in carbon-containing materials, with an organic acid (e.g., a carboxylic acid) of up to 4 carbon atoms or a benzoic acid, or a salt or ester of any of these acids, preferably acetic acid and/or one or more salts and/or one or more esters of acetic acid (i.e., one or more acetates) under conditions of temperature, pressure, and the like, that are effective to solubilize at least a portion of the carbonaceous material. Various combinations of these may also be used sequentially. Preferred agents include hydrogen peroxide, sodium hydroxide, and ethyl acetate. Sequential application of these chemicals is especially useful.

Other chemicals utilized for solubilization prior to electrical stimulation include potassium hydroxide in place of sodium hydroxide and/or a different acetate in place of ethyl acetate. The concentrations of these chemicals, as well as their relative volumes and the temperatures at which they are contacted with the coal, will vary depending upon a range of factors including the characteristics of the coal being solubilized and/or the conditions of any subterranean formation from which the coal is to be extracted.

Preferred salts or esters of acetic acid include, but are not limited to, methyl acetate, ethyl acetate, propyl acetate, isopropyl acetate, n-butyl acetate, isobutyl acetate, amyl acetate, isoamyl acetate, hexyl acetate, heptyl acetate, octyl acetate, nonanyl acetate, decyl acetate, undecyl acetate, lauryl acetate, tridecyl acetate, myristyl acetate, pentadecyl acetate, cetyl acetate, heptadecyl acetate, stearyl acetate, behenyl acetate, hexacosyl acetate, triacontyl acetate, benzyl acetate, bornyl acetate, isobornyl acetate and cyclohexyl acetate. Similar esters and salts may be formed from other carboxylic acids recited herein.

Additional solvents that can be used in conjunction with an organic acid include phosphorous acid, phosphoric acid, triethylamine, quinuclidine HCl, pyridine, acetonitrile, diethylether, acetone, dimethyl acetamide, dimethyl sulfoxide, tetrahydrothiophene, trimethylphosphine, HNO₃, EDTA, sodium salicylate, triethanolamine, 1,10-o-phenanthroline, sodium acetate, ammonium tartrate, ammonium oxalate, ammonium citrate tribasic, 2,3-dihydroxylbenzoic acid, 2,4-dihydroxylbenzoic acid, 3,4-dihydroxylbenzoic acid, 3,5-dihydroxylbenzoic acid, THF—tetrahydrofuran.

In a preferred embodiment, where the solubilization chemicals include at least two of a peroxide, a hydroxide and an acetate, more preferably where all three are utilized, the chemicals are contacted with a subterranean deposit, layer or formation either as a mixtures or sequentially, such as a sequence of injections of said chemicals. When added as a mixture, the chemicals are added together as a single composition or are added in sequence so that the mixture forms in situ. When added in sequence, each injection is optionally separated from the one before or after by injection of a suitable solvent, for example, water.

For example, one embodiment includes injecting the peroxide, followed by injecting the hydroxide, followed by injecting an acetate, each such injection separated by an injection of a volume of water followed by electrical stimulation of microbial consortia. It is to be understood that excess solvents may need to be removed from the formation before the introduction of microbial consortia that may be adversely affected by the presence of one or more of these solvents. In addition, conditions of temperature, pressure and pH that facilitate solubilization may be somewhat different from those that facilitate bioconversion.

In one embodiment, the solubilization chemicals comprise at least one peroxide, at least one hydroxide and at least one ester, preferably an acetate, together with additional chemicals, either by separate injection or injection together with a peroxide, hydroxide or acetate.

In one embodiment, the fluids introduced into the carbon-bearing deposit, such as a coalseam, contain additional solvents useful in facilitating the process of the invention, or used as part of the process of the invention, such as aromatic hydrocarbons, creosote and heavy oils. The preferred aromatic hydrocarbons include phenanthrene, chrysene, fluoranthene and pyrene, Nitrogenous ring aromatics, for example, acridine and carbazole, as well as catechol and pyrocatechol, are also suitable as solvents in the processes of the invention. Aromatics such as anthracene and fluorene may also be used. A useful solvent includes any of the foregoing, as well as mixtures, preferably a eutectic composition, thereof.

Such mixtures can usefully be dissolved in a carrier liquid, for example, a heavy oil (such a mixture being no more than about 5% to 10% of the dissolved solvent). Such solvents are most useful when heated to temperatures in the range of 80 to 400° C., preferably 80 to 300° C., more preferably 100 to 250° C., and most preferably at least about 150° C. Temperatures higher than about 400° C. are less advantageous.

In preferred embodiments, the contacting with one or more of the chemicals recited herein for solubilization is effected at temperatures in the range 0 to 300° C., including temperatures of 0 up to 200° C., preferably at a temperature of 10 to 200° C.

In other preferred embodiments, the contacting with one or more of the chemicals recited herein for solubilization is effected at a variety of pH conditions that include pH ranges 2 to 12, 3 to 11, 5 to 10, and the like, or can lie in the acid or alkaline range, such as 1 to 6, 2 to 5, or 3 to 4, or in the range 8 to 13, or 9 to 12, or 10 to 11.

It is understood that, following such solubilization methods, conditions (especially temperature and pH) may need to be changed moderately or even drastically to facilitate the action of microbial consortia present in hollow spaces of the formation or that have been intentionally introduced into the formation so as to achieve bioconversion of solubilized products into fuels, such as methane and other fuel precursors, such as fatty acids and hydrocarbons.

Materials present in the injected solubilization fluids also include other esters, such as phosphite esters. An ester of phosphite is a type of chemical compound with the general structure P(OR)₃. Phosphite esters can be considered as esters of phosphorous acid, H₃PO₃. A simple phosphite ester is trimethylphosphite, P(OCH₃)₃. Phosphate esters can be considered as esters of phosphoric acid. Since orthophosphoric acid has three —OH groups, it can esterify with one, two, or three alcohol molecules to form a mono-, di-, or triester. Chemical compounds such as esters of phosphite and phosphate, or an oxoacid ester of phosphorus, or a thioacid ester of phosphorus; or a mixture of an oxoacid of phosphorus and an alcohol, or a mixture of an thioacid of phosphorus and an alcohol, react with carbon-bearing molecules to break carbon bonds within the molecules and add hydrogen molecules to these carbon-bearing molecules, to thereby yield a range of smaller carbon-bearing molecules, such as carbon monoxide, carbon dioxide and volatile fatty acids, which are in turn more amenable to bioconversion by methanogenic microbial consortia to methane and other useful hydrocarbons. The reaction products produced from reaction of the introduced oxoacid ester of phosphorus or the thioacid ester of phosphorus; or the mixture of an oxoacid of phosphorus and an alcohol or the mixture of a thioacid of phosphorus and an alcohol; with coal may stimulate a methanogenic microbiological consortium in the subterranean formation to start producing, or increase production of, methane and other useful products.

Where carbonaceous material is treated to solubilize at least a portion of the material, the bioconversion is effected in conjunction with such treating or occurs after such treating, such as where solubilizing solvents have been extracted from the formation before introducing nutrients to facilitate the bioconversion.

In embodiments that utilize a salt or an ester of acetic acid, including, but not limited to, acetate salts and esters of alcohols and acetic acid, said salts or esters are optionally mixed with water, preferably in admixture with water. Such acetate may also be an ester. Where such chemicals are introduced into a formation to solubilize at least a portion of the carbonaceous material therein, it may be advantageous to inject water ahead of the salt or ester.

The present invention also contemplates the bioconversion of carbon-bearing materials in subterranean formations to methane and other useful hydrocarbons by first treating the subterranean formation with a solution containing at least one of an oxoacid ester of phosphorus or a thioacid ester of phosphorus; one or more aromatic alcohols; and one or more other chemical compounds/chemical entities selected from the group consisting of: hydrogen, carboxylic acids, esters of carboxylic acids, salts of carboxylic acids, oxoacids of phosphorus, salts of oxoacids of phosphorus, vitamins, minerals, mineral salts, metals, and yeast extracts.

Useful combinations of temperature and pH are contemplated by the invention and those skilled in the art are believed well able to determine, without any undue experimentation, the conditions, or combinations of such conditions, best suited for treatment of any particular carbonaceous material or deposit. Use of these combinations with varying ranges of pressure are also contemplated.

What is claimed is:

1. A bioconversion process, comprising: introducing electrical energy into a carbonaceous formation to stimulate microbes or microbial consortia in said deposit and recovering a formed product from the formation, wherein said product is a fuel or a fuel precursor.

2. The method of claim 1, wherein said microbe is a microbe indigenous to said formation.

3. The method of claim 1, wherein said microbe is an exogenous microbe introduced into said formation prior to introducing said electrical energy.

4. The method of claim 1, wherein a fluid is also introduced into said carbon bearing deposit.

5. The method of claim 4, wherein the fluid contains nutrients that promote or support the growth of microbes present in said carbon bearing deposit.

6. The method of claim 4, wherein the fluid contains chemicals that solubilize the coal.

7. The method of claim 4, wherein the fluid contains microbes capable of bioconverting carbon-bearing material to fuels or fuel precursors.

8. The method of claim 1, wherein the delivery of electrical energy is continuous.

9. The method of claim 1, wherein the fuel is methane.

10. The method of claim 1, wherein the microbe is a methanogen.

11. The method of claim 1, wherein wellbores are extended from the surface into the carbon bearing deposit.

12. The method of claim 11, wherein said wellbores comprise one or more of injection wells, production wells, and electrical energy delivery wells.

13. The method of claim 1, wherein said carbonaceous material is coal.

14. The method of claim 13, wherein said coal is bituminous coal.

15. The method of claim 1, wherein carbon dioxide is added to water, nutrients, chemicals and gases that are introduced into said carbonaceous formation and is converted into methane by methanogenic consortia in the coalseam and then produced from the coalseam.

16. The method of claim 1, wherein said carbonaceous material is contacted with a solvent to solubilize at least a portion of said carbonaceous material prior to introducing electrical energy into said formation.

17. The method of claim 16, wherein said solvent is a member selected from a salt, an ester, a peroxide and a hydroxide.

18. The method of claim 17, wherein said solvent is an acetate.

19. The method of claim 16, wherein said carbonaceous material is coal.

20. The method of claim 19, wherein said coal is bituminous coal.
